(19) United States
(12) Patent Application Publication
(10) Pub. No.: US 2007/0214007 A1
(43) Pub. Date: Sep. 13, 2007

(54) Case-finding systems and methods

(75) Inventor: Dani Hackner, Sherman Oaks, CA (US)

Correspondence Address: JONES DAY, 555 SOUTH FLOWER STREET FIFTIETH FLOOR, LOS ANGELES, CA, 90071, US

(73) Assignee: Cedars-Sinai Medical Center

(21) Appl. No.: 11/373,568

(22) Filed: Mar. 10, 2006

(51) Int. Cl. G06Q 10/00; A61B 5/00

(52) U.S. Cl. 705/2; 600/300

(57) ABSTRACT

The present invention relates to quality improvement in patient care. In particular, the present invention relates to a rapid, easily computer-implemented case-finding algorithm to identify patients with target clinical conditions.

### FIELD OF THE INVENTION

The present invention relates to quality improvement in patient care. In particular, the present invention relates to case-finding systems and methods to rapidly identify and track patients with target clinical conditions.

### BACKGROUND OF THE INVENTION

Rapid identification and concurrent tracking of large groups of patients with target clinical conditions is an urgent priority for health care providers. Over the past years, studies have shown that quality problems associated with health care may be responsible for thousands of deaths each year (Romano, N. Engl. J. Med 353:3, 2005). While there has been a proliferation of clinical practice guidelines designed to improve the quality of care for numerous conditions, there currently exists no practical method to implement these guidelines at an organizational level. Indeed, the critical first step in implementing these guidelines is identifying those patients to whom the guidelines pertain, remains a largely unsolved problem in health care. Most attempts at concurrent patient tracking to improve patient outcomes have proven impractical or ineffective, as they required either time- and labor-intensive manual screening of admission diagnoses or analysis of discharge diagnoses in the administrative data set for performance, resulting in belated discovery of coding problems and performance issues at a point where they were no longer remediable. The latter efforts especially highlight the insensitivity of concurrent efforts and the futility of post-hoc approaches to performance improvement.

Until recently, physician computer order entry was considered the panacea for performance, utilization, and quality improvement. It was anticipated that physicians would embrace the program, could provide diagnostic information concurrently, would now select guidelines/pathways and order sets, and could follow explicit instructions from alerts generated. However, perceived speed of entry issues, lack of apparent added value, repeated or nonsense alerts, and dissatisfaction with unwieldy order lists and order sets have prevented the widespread implementation of such systems and prompted renewed efforts to identify methods of enhancing care that do not rely on physician feedback in minutes or hours, force functions, or immediate alerts.

Although medical records are now almost universally computerized, there currently still exists a need for a case-finding and patient tracking method that is able to use the clinical information contained within these records. Thus, a system that adds value without asking physicians to enter their diagnosis or choose a diagnosis-specific order set, that is able to identify the likelihood of a particular diagnosis by gauging certain physician practices, and that can be initiated the moment any patient data enters the system, thereby enabling performance improvement staff to influence patient care prior to hospital discharge, would present a major improvement in the art.

### SUMMARY OF THE INVENTION

The present invention provides a novel system and method of rapidly identifying patients with target clinical conditions. The advantages of the present invention include its ability to identify patients with suspected clinical conditions using existing and readily available data sources, its ability to generate lists of patients with suspected target clinical conditions continuously, rapidly, and in real time, thereby enabling medical care professionals to monitor and treat specific groups of patients, and to comply with mandated treatment guidelines. The present invention is able to extract meaningful diagnostic information from existing and readily available data sources to sort patients into groups with suspected target clinical conditions. The present invention can be used to identify symptomatic, as well as asymptomatic patients with one or more target clinical conditions within minutes after a patient is admitted to the hospital or other facility.

In a preferred embodiment, the present invention features a computer-implemented system and method of identifying and/or tracking patients with target clinical conditions, comprising scoring terms in diagnostic or complaint text fields; scoring medications; scoring laboratory test data; and sorting scored patient data into groups of suspected clinical conditions. Thus, patients are identified in real time by the system and method of the present invention through a combination of computer-screening patient data and scoring terms from text fields including chief complaints, comments, and diagnostic information, computer-screening and scoring medications from an electronic flat file or Medication Administration Record, and screening and scoring laboratory values or other test data.

While screening data from the medications prescribed, the system and method of the present invention counts certain classes of medications per patient and tabulates different classes of medications into scores, which will be combined with the scores obtained from screening chief complaints/diagnosis and laboratory or other test data. The system and method of the present invention also computer-screens patient data for chief complaints or diagnoses and checks them against a set of standard terms and/or partial terms. The system and method of the present invention further computer-screens patient data for laboratory or other test values, using threshold values to sort patients into certain diagnoses, exclude certain diagnoses for patients, or combine them with additional patient information, including medication or diagnosis scores, to sort patients into or exclude them from suspected clinical conditions. Lastly, the system and method of the present invention is able to perform all functions concurrently to create lists of patients with one or more suspected target clinical conditions.

### BRIEF DESCRIPTION OF THE DRAWINGS

FIG. 1 shows a flow chart of a representative case finding algorithm.

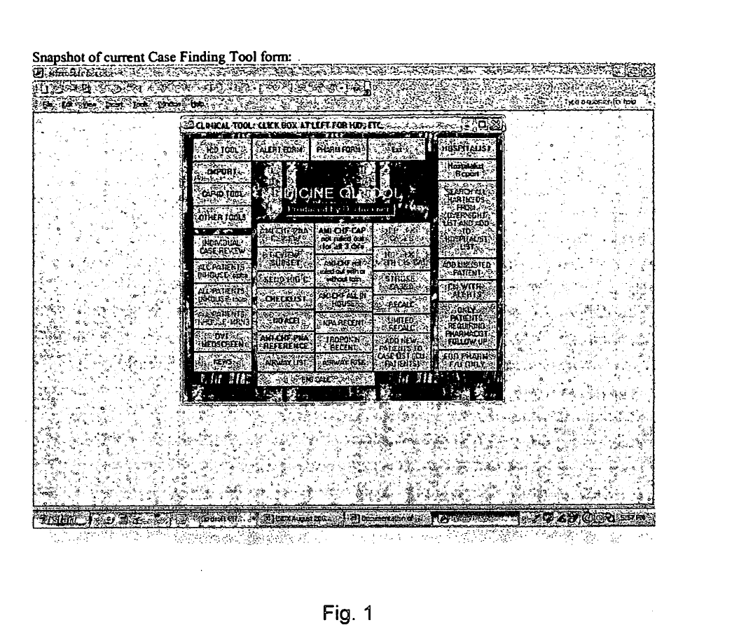

FIG. 2 shows a snapshot of a representative computer-implemented case finding program.

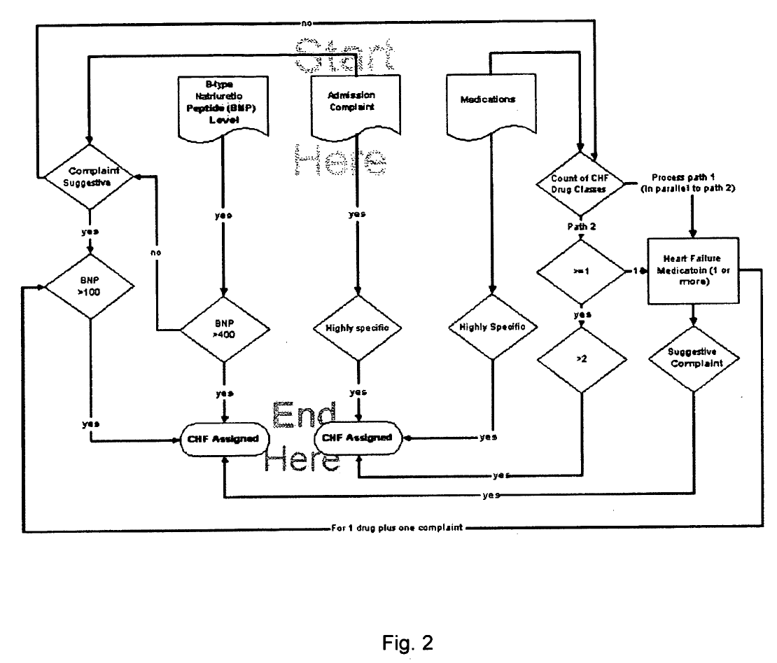

### DETAILED DESCRIPTION OF THE INVENTION

The present invention provides a novel system and method of rapidly identifying and/or tracking patients with clinical conditions. The advantages of the present invention include its ability to identify patients with suspected clinical conditions using existing and readily available data sources, its ability to generate lists of patients with suspected clinical conditions continuously, rapidly, and in real time, thereby enabling medical care professionals to monitor and treat specific groups of patients, and to comply with mandated treatment guidelines. The present invention is able to extract meaningful diagnostic information from existing and readily available data sources to sort patients into groups with suspected clinical conditions. The present invention can be used to identify symptomatic, as well as asymptomatic patients with one or more target clinical conditions within minutes after a patient is admitted to the hospital or other facility.

In a preferred embodiment, the present invention identifies and/or tracks patients with suspected target clinical conditions in real time through a combination of computer-screening patient data and scoring terms from text fields including chief complaints, comments, and diagnostic information, computer-screening and scoring medications from an electronic flat file or Medication Administration Record, and screening and scoring laboratory values or other test data.

While screening data from the medications prescribed, the system and method of the present invention counts certain classes of medications per patient and tabulates different classes of medications into scores, which are then combined with the scores obtained from screening chief complaints/diagnosis and laboratory or other test data. The system and method of the present invention also computer-screens patient data for chief complaints or diagnoses and checks them against a set of standard terms and/or partial terms. The system and method of the present invention further computer-screens patient data for laboratory or other test values, using threshold values to sort patients into certain diagnoses, exclude certain diagnoses for patients, or combine them with additional patient information, including medication or diagnosis scores, to sort patients into or exclude them from suspected clinical conditions. Lastly, the system and method of the present invention is able to perform all functions concurrently to create lists of patients with one or more suspected clinical conditions.

The following is a detailed summary of one representative use of several data sources as part of a combined electronic algorithm to identify heart failure patients, community acquired pneumonia patients, and acute myocardial infarction patients. It should be noted that heart failure, community-acquired pneumonia, and acute myocardial infarction are merely exemplary target clinical conditions which can be identified by the system and method of the present invention and that the system and method disclosed herein can be readily adapted to identify any additional target conditions. Data sources useful in conjunction with the present invention include (1) text fields including comments/diagnostic information, (2) medications from an electronic flat file or Medication Administration Record, and (3) laboratory data (such as B-type Natriuretic Peptide Assay levels or Troponin I levels). Strategies include multiple thresholds for subpopulations, combinations of lower specificity variables to improve predictive value, natural language processing (partial matches of keywords), and ranking by medication class and utilization.

Identification of New Symptomatic Heart Failure Patients: these patients may present to emergency department, monitored areas, (floor areas occasionally), and intensive care units with symptomatic heart failure. They fall into two categories, those who are diagnostic dilemmas and those who have an obvious diagnosis, based on examination, imaging, and history. For those cases with admitting complaint/diagnosis highly predictive of heart failure, the text field is all that is needed to assign as likely heart failure. However, while a clinician will have no challenge recognizing the complaint, a computerized algorithm must be sensitive to variations in clinician terminology. Until the time when all complaints are mapped to SNOWMED, the Systematized Nomenclature of Medicine Clinical Terms developed by the College of American Pathologists. There are however relatively simple approaches to this problem for a single diagnosis.

The key to capturing complaints, entered at triage by a nurse and transmitted from location to location is recognizing the errors in text entry. While most emergency triage personnel use a discrete drop-down list, there is the potential for free text or use of alternate, similar complaints. A natural language processing algorithm matches parts of key diagnostic phrases to assign these patients to the CHF (congestive heart failure) group.

Examples of highly predictive complaints used by the system and method of the present invention to identify suspected heart failure patients include terms such as heart failure (e.g. “*heart*” and “*failure*), pulmonary edema (e.g. “*pulmonary edema*”), and congestive heart failure (e.g. “*CHF*” or “*C H F*”).

For cases with admitting complaints that are highly suggestive, but with major alternatives in the differential diagnosis, a second variable is used. Examples of suggestive complaints include, for example, dyspnea, shortness of breath (e.g. “*SOB*” or “*shortness*”), congestion (e.g. “*congest*”), cardiomyopathy (e.g. “*cardio*”), edema, weakness, volume overload, renal insufficiency, and atrial fibrillation. The second variable that can be used with the system and method of the present invention to identify the actual diagnosis of heart failure is a B-type Natriuretic Peptide (BNP) Assay level of greater than 100 ng/dl. This assay provides high sensitivity, but with relatively low specificity. If BNP levels are not elevated, the present invention rules out the diagnosis in clinical terms, despite the admitting complaint. However, elevated BNP levels, together with a suggestive admitting complaint, provide improved specificity. Thus, symptomatic patients with signs of left heart ‘stretch’ or ‘strain’ causing elevated natriuretic peptide levels or characteristic complaints, are identified as heart failure patients. The system and method of the present invention does not rely on chest x-ray findings which may or may not correlate with symptoms. In addition, the present invention takes advantage of the pattern of practice which has led easily identifiable cases to be labeled with specific diagnoses/complaints and difficult to sort out cases to have natriuretic peptide levels drawn. In these situations the challenge is not using the information, but using it in real time, which is now made possible by the present invention.

Identification of asymptomatic patients or patients not yet treated for heart failure during a workup: these patients may not have been identified at triage initially or a suggestive complaint may not be captured by clinicians or computer systems. These patients fall into two broad categories, treated and untreated. For untreated patients with physiologic signs of heart failure but not necessarily presenting initially so, the present invention takes another strategy. For cases presenting with heart failure after time of admission or showing physiologic signs along with another diagnosis, the present invention takes advantage of the greater specificity of the natriuretic peptide (BNP) assay at a higher threshold. While this approach is less sensitive, it captures a group of patients that lack an identifying admission diagnosis or have not yet been started on heart failure medications. Thus, a useful threshold for B-Type Natriuretic Peptide levels is greater than 400 ng/dl. as lower levels have been used as criterion for discharge from hospital and have low specificity. No corroborating information is available or is needed in this instance, which focuses on capturing specific heart failure cases. While there are cases of right heart strain, pulmonary hypertension, pulmonary embolism and others that may fall into this group, the low incidence of these entities does not make this a practical problem. The key to this approach is that it enables identification prior to medications appearing that would narrow the diagnosis further, allowing earlier focusing of the diagnosis and narrowing the patient group.

Treated patients with or without physiologic signs of heart failure can be separated into three groups: (1) patients with acute heart failure and suggestive medications, (2) patients with acute heart failure and very specific heart failure medications, and (3) patients with chronic heart failure. Patients with treated, possible heart failure, and suggestive medications: acute heart failure medication is not sufficient to determine that a clinician has treated the patient. However, as the number of medications increase, the specificity improves. With two or more heart failure medications the system and method of the present invention can combine this moderate threshold with another sensitive indicator, i.e. natriuretic peptide levels. The chosen medication threshold can be set at equal to or greater than two heart failure medication classes. These medications are mapped to classes with brand names and generics included. Patients receiving two medications from a single class (as in the case of a switch) in one calendar day are only counted as receiving one medication class. Useful classes of heart failure medications for the purposes of the present invention include: angiotensin converting enzyme class (e.g. benazepril), beta blocker class (e.g. metoprolol, atenolol), cardiac glycoside class (digoxin, Lanoxin), angiotensin receptor blocker class (e.g. candesartan), and heart failure diuretic class (e.g. furosemide). Medications considered but not included due to confounding hypertension cases include angiotensin receptor blockers. B-type Natriuretic Peptide level threshold is set at greater than 100 ng/dl, as values below this level rule out acute heart failure in practical terms. This algorithm can be used in addition, if BNP levels are mildly elevated and medications are not present.

Patients with treated, acute heart failure, and specific medications: certain medications have been developed and marketed specifically for heart failure. Examples include Carvedilol, a beta blocker, with evidence of greater mortality reduction than some others in the class for heart failure patients. Others identify a group of patients with right or left heart dysfunction or rhythm problems. The present invention sets a medications threshold of one or more specific medication, including Carvedilol (selective beta blocker), Digoxin (inotrope), Aldactone (aldosterone antagonist), and Eplerenone (aldosterone antagonist). Only one class of any of these medications is needed to combine with a natriuretic peptide level that rules out ‘no heart failure’. B-type Natriuretic Peptide level threshold is set at greater than 100 ng/dl, as a low BNP level rules out acute heart failure in practical terms. In addition, if BNP levels are mildly elevated, but a single specific medication is not present, the patient must be captured by one of the other algorithms in order to be included.

Identification of treated, chronic heart failure patients. This group of patients may have an acute exacerbation, which might be picked up by other algorithms, or they may not have natriuretic peptide levels drawn or complaints assigned. The value of the algorithm of the present invention is that it identifies early a case awaiting workup but empirically treated as heart failure. The medication threshold is set at equal to or greater than three medications selected from the following groups: angiotensin converting enzyme class (e.g. benazepril), beta blocker class (e.g. metoprolol, atenolol), cardiac glycoside class (digoxin, Lanoxin), angiotensin receptor blocker (e.g. candesartan), and heart failure diuretic (e.g. furosemide).

Identification of acute myocardial infarction patients: this algorithm uses a highly sensitive approach. Because troponin I levels reflect muscle injury during myocardial infarction, a troponin I level threshold is set at greater than 0.4 mg/dl within 24 hours. Troponin I levels may even be elevated without obvious signs on routine electrocardiogram, which is useful but not sensitive enough. Troponin may not be elevated early when an acute myocardial infarction receives very early intervention (within minutes). Some pitfalls associated with the use of troponin I levels include: troponin I may rise after cardiac procedures such as percutaneous coronary interventions, balloon angioplasty, or stent; small Troponin I rises may reflect strain or even heart failure, and secondary myocardial infarctions may occur from issues such as low flow states, bleeding, trauma, or spasm. Some of these may not be picked up if Troponin I within 24 hours is used. However, some patients may present late to hospital and history is essential. While troponin I levels are a sensitive marker, due to specificity limitations, use should be limited to early warnings and point of care reminders. Thus, early warnings should not be firm or rigid. Further, Troponin I-based tracking should be coupled with full review after the fact of all cases assigned a Principal Diagnosis of heart failure and especially of cases with marginal Troponin I levels.

Identification of Community Acquired Pneumonia Patients: a first approach is the high likelihood diagnosis. For those cases with admitting complaints highly predictive, the complaint is all that is needed initially. However, while a clinician will have no challenge recognizing the complaint, a computerized algorithm must be sensitive to variations in clinician terminology. Until the time when all complaints are mapped to SNOWMED vocabularies, this problem will persist. The present invention provides some relatively simple approaches to this problem for a single diagnosis. The complaint list can be screened for the following terms: 1. “Cough”+“Fever” (in a patient admitted to hospital), and 2. Pneumonia or variation (like “pneumon*”). While this is by no means specific for pneumonia, in the absence of medication information, radiographic information, or other historical information, it enables a small subset that is very likely to have pneumonia to be identified. The second approach is identification by suggestive complaint plus medication, as for several presenting complaints, the complaint by itself is not enough to establish a high likelihood of pneumonia. The complaint terms are chosen from the following complaint/diagnostic list: 1. Cough, 2. Bronch, 3. CAP, 4. Pleuritic pain, 5. Fever (alone). These complaints need corroborating medications, to confirm a plan underway for pneumonia treatment. Corroborating medications are chosen from the following list: 1. Cefotaxime, 2. Cefuroxime, 3. Azithromycin, 4. Levofloxacin, and others as indicated in the American Thoracic and IDSA guideline recommendations. It should be noted that regimens used for in-hospital pneumonias and nursing home pneumonias are not included. The medication threshold set by the present invention is as follows: for most complaints, the threshold is set at equal to or greater than one community acquired pneumonia medication. For fever, at least two common community acquired pneumonia medications are required.

While helpful for identifying pneumonia patients, the algorithm does not identify pneumonia patients prior to medication administration in most cases. There is little evidence that without radiographic information, examination and complete histories that this can be done automatically. However, the identification of community acquired pneumonia cases within a day offers benefits in terms of medication utilization, vaccination, oxygenation assessment and other measures that can be initiated or tracked based on the identification strategy. Most importantly, used in combination with heart failure strategies, this part of the algorithm can be invaluable in separating the diagnosis, often confusing to clinicians. It may be used to trigger early coding as well.

Thus, combining tracking with heart failure allows natriuretic levels to improve the specificity of the CAP (community-acquired pneumonia) diagnosis by using B-type Natriuretic Levels lower than 100 ng/dl.

Use of electronic identification in case finding algorithms: one of the advantages provided by the present invention is the early identification of high likelihood cases. The present invention provides early use of a high sensitivity algorithm, enabling clinicians or repeat electronic reviews to add to specificity. When this occurs, time is on the side of such a protocol. The present exemplary algorithms identify cases by hospital from day one using only medication lists, admitting complaint and laboratory data. Even for the algorithms with greater sensitivity and lower specificity, such as those employing B-type Natriuretic levels of 100 (in combination with other data), early identification leads to time being on the clinician's side. If one narrows 550 inpatients to a group of 30 on hospital day one in an automated algorithm, the identifying diagnosis will be of high specificity because manageable and in some cases a chart review that takes minutes becomes trivial if begun on day one of a 4-6 day length of stay.

The present invention provides an added value step by immediately flagging very high likelihood cases. Cases with highly specific complaints, medications, or assay levels do not need high levels of corroborating data. These cases will be fewer and the strategy is not sensitive, but alongside the other strategies, it reduces the load of work for second reviews or corrections. The present invention further provides an automatic mechanism that interprets the importance of medications, which is highly useful, as physicians indicate what they are thinking by how they prescribe medications. This represents a significant advantage over asking physicians to assign diagnoses, since this requires electronic entry by physicians or chart abstraction, both of which are time consuming and fraught with error early in the hospital stay especially. With the system and method of the present invention, on the other hand, physicians are telling the system what they are prioritizing based on their prescribing.

Moreover, it is unclear in most cases whether a chronic heart failure case or chronic ischemic disease will be coded as acute heart failure or acute myocardial infarction until close to time of discharge. At that juncture, highly specific case finding has value if coupled with early sensitive case finding which allow point of care interventions, reminders, and assistance.

The integrated algorithms provided by the present invention, without the necessity for a computerized medical record, allow the implementation of clinical reminder systems for managing chronic illness, thereby raising reportable quality of care, allow the identification of cases for re-coding, and provide medication utilization reminders.

### EXAMPLES

The following examples are provided to better illustrate the claimed invention and are not to be interpreted as limiting the scope of the invention. To the extent that specific medications or terms are mentioned, it is merely for purposes of illustration and is not intended to limit the invention. Based on the present inventive concept one skilled in the art can readily develop equivalent means without the exercise of inventive capacity and without departing from the scope of the invention. It will be understood that many variations can be made in the procedures herein described while still remaining within the bounds of the present invention.

### Example 1

Identification of New Symptomatic Heart Failure Patients

Rationale: These patients may present to emergency department, monitored areas, (floor areas occasionally), and intensive care units with symptomatic heart failure. They fall into two categories, those who are diagnostic dilemmas and those who have an obvious diagnosis, based on examination, imaging, and history.

A. For those cases with admitting complaint/diagnosis highly predictive of heart failure, the text field is all that is needed to assign as likely heart failure. However, while a clinician will have no challenge recognizing the complaint, a computerized algorithm must be sensitive to variations in clinician terminology. Until the time when all complaints are mapped to SNOWMED vocabularies, this problem will persist. There are however relatively simple approaches to this problem for a single diagnosis.
1. The key to capturing complaints, entered at triage by a nurse and transmitted from location to location is recognizing the errors in text entry. While most emergency triage personnel use a discrete drop-down list, there is the potential for free text or use of alternate, similar complaints. A natural language processing algorithm matches parts of key diagnostic phrases to assign these patients to the CHF group.
2. Highly predictive complaints
   a. Heart Failure (e.g. like “*heart*” and like “*failure*)
   b. Pulmonary Edema (e.g. like “*pulmonary edema*”)
   c. Congestive Heart Failure (e.g. like “*CHF*” or like “*C H F*”)

B. For those cases with admitting complaints highly suggestive, but with major alternatives in the differential diagnosis, a second variable is used.
1. Examples of Suggestive Complaints
   a. Dyspnea
   b. Shortness of breath (e.g. like “*SOB*” or like “*shortness*”)
   c. Congestion (e.g. like “*congest*”)
   d. Cardiomyopathy (e.g. like “*cardio*”)
   e. Edema
   f. Weakness
   g. Volume overload
   h. Renal insufficiency
   i. Atrial fibrillation
2. Second Variable
   a. B-type Natriuretic Peptide Assay level>100 ng/dl
   b. Highly sensitive, but with low sensitivity
   c. If not elevated, rules out the diagnosis in clinical terms, despite the admitting complaint
   d. Together with a suggestive admitting complaint, specificity improves

C. In summary, symptomatic patients with signs of left heart ‘stretch’ or ‘strain’ causing elevated natriuretic peptide levels or characteristic complaints, new patients are identified. It does not rely on chest x-ray findings which may or may not correlate with symptoms. In addition, we have taken advantage of the pattern of practice which has led easily identifiable cases to be labeled with specific diagnoses/complaints and difficult to sort out cases to have natriuretic peptide levels drawn. In these situations the challenge is not using the information, but using it in real time.

### Example 2

Identification of asymptomatic patients or patients not yet treated for heart failure during a workup. These patients may not have been identified at triage initially or a suggestive complaint may not be captured by clinicians or computer systems. These patients fall into two broad categories, treated and untreated.

A. For untreated patients with physiologic signs of heart failure but not necessarily presenting initially so, another strategy is taken. For cases presenting with heart failure after time of admission or showing physiologic signs along with another diagnosis, advantage is taken of the greater specificity of the natriuretic peptide assay at a higher threshold. While this approach is less sensitive, it captures a group of patients that lack an identifying admission diagnosis or have not yet been started on heart failure medications.
1. Natriuretic Peptide Assay levels
   a. B-Type Natriuretic Peptide >400 ng/dl
   b. Rationale: lower levels have been used as criterion for discharge from hospital and have low specificity
   c. No corroborating information is available or is needed in this instance, which focuses on capturing specific heart failure cases.
   d. There are cases of right heart strain, pulmonary hypertension, pulmonary embolism and others that may fall into this group, however, the low incidence of these entities does not make this a practical problem. The key to this approach is that it enables identification prior to medications appearing that would narrow the diagnosis further, allowing earlier focusing of the diagnosis and narrowing the patient group.

B. For treated patients with or without physiologic signs of heart failure, these patients fall into three groups: (1) patients with acute heart failure and suggestive medications, (2) patients with acute heart failure and very specific heart failure medications, and (3) patients with chronic heart failure.
1. Treated, possible heart failure, and suggestive medications.
   a. Acute heart failure medication is not sufficient to determine that a clinician has treated the patient. However, as the number of medications increase, the specificity improves. With two or more heart failure medications we can combine this moderate threshold with another sensitive indicator, natriuretic peptide levels.
   b. Chosen medication threshold: ≧2 heart failure medication classes. These medications are mapped to classes with brand names and generics included. Patients receiving two medications from a single class (as in the case of a switch) in one calendar day are only counted as receiving one medication class.
      i. Angiotensin Converting Enzyme class (e.g. benazepril)
      ii. Beta blocker class (e.g. metoprolol, atenolol)
      iii. Cardiac glycoside class (digoxin, Lanoxin)
      iv. Angiotensin Receptor Blocker class (e.g. candesartan)
      v. Heart failure diuretic (e.g. furosemide)
   c. Medication considered but not included due to confounding hypertension cases
      i. Angiotensin Receptor Blockers
   d. B-type Natriuretic Peptide Level of >100 ng/dl
      i. This level rules out acute heart failure in practical terms if BNP not elevated.
      ii. In addition, if BNP mildly elevated and medications are not present
2. Treated, acute heart failure, and specific medications
   a. Certain medications have been developed and marketed specifically for heart failure. Examples include Carvedilol, a beta blocker, with evidence of greater mortality reduction than some others in the class for heart failure patients. Others identify a group of patients with right or left heart dysfunction or rhythm problems.
   b. Medications threshold: 1 or more specific medication
      i. Carvedilol (selective beta blocker)
      ii. Digoxin (inotrope)
      iii. Aldactone (aldosterone antagonist)
      iv. Eplerenone (aldosterone antagonist)
   c. Only one class is needed to combine with a natriuretic peptide level that rules out ‘no heart failure’
   d. B-type Natriuretic Peptide Level of >100 ng/dl
      i. A low level rules out acute heart failure in practical terms.
      ii. In addition, if BNP mildly elevated and a single specific medication is not present, other algorithms must capture in order to include
3. Treated, chronic heart failure. These patients may have an acute exacerbation, which might be picked up by other algorithms, or they may not have natriuretic peptide levels drawn or complaints assigned. The value of this algorithm is that it identifies early a case awaiting workup but empirically treated as heart failure.
   a. Medication threshold: ≧3
      i. Angiotensin Converting Enzyme class (e.g. benazepril)
      ii. Beta blocker class (e.g. metoprolol, atenolol)
      iii. Cardiac glycoside class (digoxin, Lanoxin)
      iv. Angiotensin receptor blocker (e.g. candesartan)
      v. Heart failure diuretic (e.g. furosemide)

### Example 3

Identification of Acute Myocardial Infarction Patients

A. This algorithm uses a highly sensitive approach
1. Troponin I threshold: >0.4 mg/dl within 24 hours
   a. This may even be elevated without obvious signs on routine electrocardiogram, which is useful but not sensitive enough.
   b. It may not be elevated early when an acute myocardial infarction receives very early intervention (within minutes)
2. Rationale: Troponin I reflects muscle injury during myocardial infarction
3. Pitfalls:
   a. Troponin I may rise after cardiac procedures such as percutaneous coronary interventions, balloon angioplasty, or stent.
   b. Small Troponin I rises may reflect strain or even heart failure.
   c. Secondary myocardial infarctions may occur from issues such as low flow states, bleeding, trauma, or spasm. Some of these may not be picked up if Troponin I within 24 hours is used. However, some patients may present late to hospital and history is essential.
4. While a sensitive marker, due to specificity limitations, use should be limited to early warnings and point of care reminders.
   a. Early warnings should not be firm or rigid
   b. Troponin I-based tracking should be coupled with full review after the fact of all cases assigned Principal Diagnosis of heart failure and especially cases with marginal Troponin I levels.

### Example 4

Identification of Community Acquired Pneumonia Patients

A. By high likelihood diagnosis
1. For those cases with admitting complaints highly predictive, the complaint is all that is needed initially. However, while a clinician will have no challenge recognizing the complaint, a computerized algorithm must be sensitive to variations in clinician terminology. Until the time when all complaints are mapped to SNOWMED vocabularies, this problem will persist. There are however relatively simple approaches to this problem for a single diagnosis.
   a. Complaint list:
      i. “Cough”+“Fever” (in a patient admitted to hospital)
      ii. Pneumonia or variation (like “pneumon*”)
   b. This is by no means specific for pneumonia, but in the absence of medication information, radiographic information, or other historical information, it enables a small subset that is very likely to have pneumonia to be identified

B. By suggestive complaint plus medication.
1. For several presenting complaints, this is not enough to establish a high likelihood of pneumonia
   a. Complaint/diagnostic list
      i. Cough
      ii. Bronch
      iii. CAP
      iv. Pleuritic pain
      v. Fever (alone)
   b. These need corroborating medications, to confirm a plan underway for pneumonia treatment
   c. Medications:
      i. Cefotaxime
      ii. Cefuroxime
      iii. Azithromycin
      iv. Levofloxacin
      v. And others per American Thoracic and IDSA guideline recommendations
      vi. Regimens used for in-hospital pneumonias and nursing home pneumonias are not included
   d. Medication threshold
      i. For most complaints, ≧1 community acquired pneumonia medication
      ii. For fever, at least 2 common community acquired pneumonia medications
      iii. While helpful for identifying pneumonia patients, the algorithm does not identify pneumonia patients prior to medication administration in most cases. There is little evidence that without radiographic information, examination and complete histories that this can be done automatically.
      iv. However, the identification of community acquired pneumonia cases within a day offers benefits in terms of medication utilization, vaccination, oxygenation assessment and other measures that can be initiated or tracked based on the identification strategy.
      v. Most importantly, used in combination with heart failure strategies, it can be invaluable in separating the diagnosis, often confusing to clinicians.
         1) It may be used to trigger early coding as well
         2) Combining tracking with heart failure allows natriuretic levels to improve the specificity of the CAP diagnosis by using B-type Natriuretic Levels lower than 100 ng/dl.

### Example 5

This example shows several algorithms in SQL form.

AMI-CHF—PNA2_{—}18

This algorithm pools multiple messages generated by subalgorithms (message 1 is for heart failure, message 2 is a heart attack message, and message 3 is for pneumonia). It looks at all cases labeled in two lists call hmcw and trackall. Hmcw includes all pharmacy data and lab data after import. Trackall includes tracer information such as cases previously seen and not included (“secondary” or “ruled out”). Trackall is a running log that winnows down new lists. The hmcw is new raw data. Nulls imply no data. Vnpa is the natriuretic peptide value (heart failure). Vtni is the troponin i value (heart attack)

SELECT HMCW.*, TRACK_ALL.*, [ACEI]+[BETA]+[ARB]+[DIURETIC]+[CHF] AS SUMCARDS

FROM HMCW INNER JOIN TRACK_ALL ON(HMCW.ADMITDATE=TRACK_ALL.AdmitDate) AND (HMCW.MRN9=TRACK_ALL.Master)

WHERE (((HMCW.Age)>17) AND ((HMCW.VNPA)<=1 Or (HMCW.VNPA)>99) AND (Not (TRACK_ALL.MESSAGE1) Is Null) AND ((TRACK_ALL.Secondary)=False Or (TRACK_ALL.Secondary) Is Null) AND ((TRACK_ALL[Ruled Out])=False Or (TRACK_ALL. [Ruled Out]) Is Null) AND ((HMCW.CREATED)=Date( ))) OR (((HMCW.Age)>17) AND ((HMCW.CREATED)=Date( )) AND (Not (TRACK_ALL.MESSAGE2) Is Null) AND ((TRACK_ALL.AMI2nd)=False Or (TRACK_ALL.AMI2nd) Is Null)) OR (((HMCW.Age)>17) AND ((HMCW.CREATED)=Date( )) AND (Not (TRACK_ALL.MESSAGE3) Is Null) AND ((TRACK_ALL.NotPNA) Is Null Or (TRACK_ALL.NotPNA)=False)) OR (((HMCW.Age)>17) AND ((TRACK_ALL. [Ruled Out])=True) AND ((HMCW.CREATED)=Date( )) AND ((TRACK_ALL.PrinAMI)=True) AND ((TRACK_ALL.IncludeonLIst)=True)) OR (((HMCW.Age)>17) AND ((TRACK_ALL.Secondary)=True) AND ((HMCW.CREATED)=Date( )) AND ((TRACK_ALL.PrinAMI)=True) AND ((TRACK_ALL.IncludeonLIst)=True)) OR (((HMCW.Age)>17) AND ((HMCW.CREATED)=Date( )) AND ((TRACK_ALL.PrinAMI)=False Or (TRACK_ALL.PrinAMI) Is Null) AND ((TRACK_ALL.IncludeonLIst)=True) AND ((TRACK_ALL.Principal)=True)) OR (((HMCW.Age)>17) AND ((HMCW.CREATED)=Date( )) AND ((TRACK_ALL.IncludeonLIst)=True) AND ((TRACK_ALL.Principal)=True) AND ((TRACK_ALL.ProbPNA)=False Or (TRACK_ALL.ProbPNA) Is Null)) OR (((HMCW.Age)>17) AND ((HMCW.CREATED)=Date( )) AND ((TRACK_ALL.AMI2nd)=True) AND ((TRACK_ALL.IncludeonLIst)=True) AND ((TRACK_ALL.ProbPNA)=True)) OR (((HMCW.Age)>17) AND ((TRACK_ALL[Ruled Out])=True) AND ((HMCW.CREATED)=Date( )) AND ((TRACK_ALL.IncludeonLIst)=True) AND ((TRACK_ALL.ProbPNA)=True))

ORDER BY HMCW.Room;

PNA_MESSAGE

HERE IS A SUB-ALGORITHM THAT GENERATES MESSAGE3 (PNEUMONIA FLAG):

UPDATE HMCW INNER JOIN TRACK_ALL ON (TRACK_ALL.AdmitDate=HMCW.ADMITDATE) AND (HMCW.MRN9=TRACK_ALL.Master) SET TRACK_ALL.MESSAGE3=“POSSIBLE PNA.”

WHERE (((TRACK_ALL.ProbPNA)=True) AND ((TRACK_ALL.NotPNA)< >True)) OR (((TRACK_ALL.NotPNA)< >True) AND ((HMCW.PNAV)>0) AND ((HMCW.Diag1) Like “*cough*” Or (HMCW.Diag1) Like “*bronch*”)) OR (((TRACK_ALL.NotPNA)< >True) AND ((HMCW.PNAV)>0) AND ((HMCW.Diag1) Like “*PNEUMON*”)) OR (((TRACK_ALL.NotPNA)< >True) AND ((HMCW.PNAV)>0) AND ((HMCW.Diag1) Like “*CAP*”)) OR (((TRACK_ALL.NotPNA)< >True) AND ((HMCW.PNAV)>0) AND ((HMCW.Diag1) Like “*PLEURIT*”)) OR (((TRACK_ALL.NotPNA)< >True) AND ((HMCW.PNAV)>0) AND ((HMCW.Diag1) Like “*LOWER RESP*”)) OR (((TRACK_ALL.NotPNA)< >True) AND ((HMCW.Diag1) Like “*COUGH*” And (HMCW.Diag1) Like “*FEVER*”)) OR (((TRACK_ALL.NotPNA)< >True) AND ((HMCW.Diag1) Like “*PNEUMON*”) AND ((HMCW.VNPA)<100)) OR (((TRACK_ALL.NotPNA)< >True) AND ((HMCW.PNAV)>2) AND ((HMCW.Diag1) Like “*fever*”));

AMI_MESSAGE

ANOTHER SUB-ALGORITHM THAT GENERATES MESSAGE2 (HEART ATTACK FLAG):

UPDATE HMCW INNER JOIN TRACK_ALL ON(HMCW.ADMITDATE=TRACK_ALL.AdmitDate) AND (HMCW.MRN9=TRACK_ALL.Master) SET TRACK_ALL.MESSAGE2=“SUSPECT AMI.”

CHF_MESSAGE

A THIRD SUB-ALGORITHM THAT GENERATES MESSAGE 1 (HEART FAILURE FLAG):

UPDATE TRACK_ALL INNER JOIN HMCW ON (TRACK_ALL.AdmitDate=HMCW.ADMITDATE) AND (TRACK_ALL.Master=HMCW.MRN9) SET TRACK_ALL.MESSAGE1=“CONSIDER CHF.”

WHERE ((([ACEI]+[ARB]+[DIURETIC])>1) AND ((TRACK_ALL.FormonChart)=False Or (TRACK_ALL.FormonChart) Is Null) AND ((TRACK_ALL.Secondary)=False Or (TRACK_ALL.Secondary) Is Null) AND ((TRACK_ALL[Ruled Out])=False Or (TRACK_ALL[Ruled Out]) Is Null) AND ((HMCW.VNPA)>100)) OR (((TRACK_ALL.FormonChart)=False Or (TRACK_ALL.FormonChart) Is Null) AND ((TRACK_ALL.Secondary)=False Or (TRACK_ALL.Secondary) Is Null) AND ((TRACK_ALL[Ruled Out])=False Or (TRACK_ALL[Ruled Out]) Is Null) AND ((HMCW.VNPA)>100) AND (([MED1] & [MED2] & [MED3] & [MED4] & [MED5] & [MED6] & [MED7] & [MED8] & [MED9] & [MED10] & [MED11] & [MED12] & [MED13] & [MED14] & [MED15]) Like “*ALDACTONE*” Or ([MED1] & [MED2] & [MED3] & [MED4]& [MED5] & [MED6] & [MED7] & [MED8] & [MED9] & [MED10] & [MED 11] & [MED12] & [MED13] & [MED14] & [MED15]) Like “*COREG*” Or ([MED1] & [MED2] & [MED3] & [MED4] & [MED5] & [MED6] & [MED7] & [MED8] & [MED9] & [MED10] & [MED11] & [MED12] & [MED13] & [MED14] & [MED15]) Like “*CARVED*” Or ([MED1] & [MED2] & [MED3] & [MED4] & [MED5] & [MED6] & [MED7] & [MED8] & [MED9] & [MED10] & [MED 11] & [MED12] & [MED13] & [MED14] & [MED15]) Like “*digoxin*” Or ([MED1] & [MED2] & [MED3] & [MED4] & [MED5] & [MED6] & [MED7] & [MED8] & [MED9] & [MED10] & [MED 11] & [MED12] & [MED13] & [MED14] & [MED15]) Like “*lanoxin*”)) OR (((TRACK_ALL.FormonChart)=False Or (TRACK_ALL.FormonChart) Is Null) AND ((TRACK_ALL.Secondary)=False Or (TRACK_ALL.Secondary) Is Null) AND ((TRACK_ALL[Ruled Out])=False Or (TRACK_ALL[Ruled Out]) Is Null) AND ((HMCW.VNPA)>400)) OR (((TRACK_ALL.FormonChart)=False Or (TRACK_ALL.FormonChart) Is Null) AND ((TRACK_ALL.Secondary)=False Or (TRACK_ALL.Secondary) Is Null) AND ((TRACK_ALL[Ruled Out])=False Or (TRACK_ALL[Ruled Out]) Is Null) AND ((HMCW.VNPA)>100) AND ((HMCW.CHF)=1)) OR (((TRACK_ALL.FormonChart)=False Or (TRACK_ALL.FormonChart) Is Null) AND ((TRACK_ALL.Secondary)=False Or (TRACK_ALL.Secondary) Is Null) AND ((TRACK_ALL[Ruled Out])=False Or (TRACK_ALL[Ruled Out]) Is Null) AND ((HMCW.VNPA)>100) AND ((HMCW.Diag1) Like “*FIBRIL*” Or (HMCW.Diag1) Like “*FAILURE*” Or (HMCW.Diag1) Like “*VOLUME*” Or (HMCW.Diag1) Like “*RENAL INSUFFIC*”)) OR (((TRACK_ALL.FormonChart)=False Or (TRACK_ALL.FormonChart) Is Null) AND ((TRACK_ALL.Secondary)=False Or (TRACK_ALL.Secondary) Is Null) AND ((TRACK_ALL[Ruled Out])=False Or (TRACK_ALL[Ruled Out]) Is Null) AND ((HMCW.Diag1) Like “*CHF*”)) OR (((TRACK_ALL.FormonChart)=False Or (TRACK_ALL.FormonChart) Is Null) AND ((TRACK_ALL.Secondary)=False Or (TRACK_ALL.Secondary) Is Null) AND ((TRACK_ALL[Ruled Out])=False Or (TRACK_ALL[Ruled Out]) Is Null) AND ((HMCW.Diag1) Like “*CONGEST*”)) OR (((TRACK_ALL.FormonChart)=False Or (TRACK_ALL.FormonChart) Is Null) AND ((TRACK_ALL.Secondary)=False Or (TRACK_ALL.Secondary) Is Null) AND ((TRACK_ALL[Ruled Out])=False Or (TRACK_ALL[Ruled Out]) Is Null) AND ((HMCW.VNPA)>100) AND ((HMCW.Diag1) Like “*CARDIO*”)) OR (((TRACK_ALL.FormonChart)=False Or (TRACK_ALL.FormonChart) Is Null) AND ((TRACK_ALL.Secondary)=False Or (TRACK_ALL.Secondary) Is Null) AND ((TRACK_ALL[Ruled Out])=False Or (TRACK_ALL[Ruled Out]) Is Null) AND ((HMCW.Diag1) Like “*MYOPATH*”)) OR (((TRACK_ALL.FormonChart)=False Or (TRACK_ALL.FormonChart) Is Null) AND ((TRACK_ALL.Secondary)=False Or (TRACK_ALL.Secondary) Is Null) AND ((TRACK_ALL[Ruled Out])=False Or (TRACK_ALL[Ruled Out]) Is Null) AND ((HMCW.Diag1) Like “*DYSPNEA*”)) OR (((TRACK_ALL.FormonChart)=False Or (TRACK_ALL.FormonChart) Is Null) AND ((TRACK_ALL.Secondary)=False Or (TRACK_ALL.Secondary) Is Null) AND ((TRACK_ALL[Ruled Out])=False Or (TRACK_ALL[Ruled Out]) Is Null) AND ((HMCW.Diag1) Like “*SHORTNESS*”)) OR (((TRACK_ALL.FormonChart)=False Or (TRACK_ALL.FormonChart) Is Null) AND ((TRACK_ALL.Secondary)=False Or (TRACK_ALL.Secondary) Is Null) AND ((TRACK_ALL[Ruled Out])=False Or (TRACK_ALL[Ruled Out]) Is Null) AND ((HMCW.Diag1) Like “*SOB*”)) OR (((TRACK_ALL.FormonChart)=False Or (TRACK_ALL.FormonChart) Is Null) AND ((TRACK_ALL.Secondary)=False Or (TRACK_ALL.Secondary) Is Null) AND ((TRACK_ALL. [Ruled Out])=False Or (TRACK_ALL. [Ruled Out]) Is Null) AND ((HMCW.Diag1) Like “*S O B*”)) OR (((TRACK_ALL.FormonChart)=False Or (TRACK_ALL.FormonChart) Is Null) AND ((TRACK_ALL.Secondary)=False Or (TRACK_ALL.Secondary) Is Null) AND ((TRACK_ALL[Ruled Out])=False Or (TRACK_ALL[Ruled Out]) Is Null) AND ((HMCW.Diag1) Like “*PULMONARY EDEMA*”)) OR (((TRACK_ALL.FormonChart)=False Or (TRACK_ALL.FormonChart) Is Null) AND ((TRACK_ALL.Secondary)=False Or (TRACK_ALL.Secondary) Is Null) AND ((TRACK_ALL. [Ruled Out])=False Or (TRACK_ALL[Ruled Out]) Is Null) AND ((HMCW.VNPA)>100) AND ((HMCW.Diag1) Like “*EDEMA*”)) OR (((TRACK_ALL.FormonChart)=False Or (TRACK_ALL.FormonChart) Is Null) AND ((TRACK_ALL.Secondary)=False Or (TRACK_ALL.Secondary) Is Null) AND ((TRACK_ALL[Ruled Out])=False Or (TRACK_ALL. [Ruled Out]) Is Null) AND ((HMCW.VNPA)>100) AND ((HMCW.Diag1) Like “*WEAKNESS*”)) OR (((HMCW.VNPA)>100) AND ((HMCW.CHF)>0));

### Example 6

Implementation of the computerized system and method with a focus on heart failure, pneumonia, and acute myocardial infarction.

In contrast with prior approaches which focused on diagnosis within minutes or hours to a high degree of certainty (>95%), the approach of the present invention accepted higher specificity, lower sensitivity and aimed for a 12 hour window. In addition, the system and method of the present invention was designed to use commonly available data that was tolerant of wide practice variation among clinicians. Another advantageous feature was the ability to extract data from a low profile, low participation system without dependence on provider participation in identifying patients. With regard to the diagnoses in question, some principals underpinned the algorithms developed: (1) many patients thought to have heart failure also need evaluation for the major competing diagnosis of pneumonia; (2) many patients with small heart attacks may ultimately leave the hospital with a discharge diagnosis of heart failure, or vice versa; (3) the algorithm need not achieve 100% specificity but must narrow a daily patient population as large as 500-850 patients down to a manageable group of 30-120 daily. Without attempting to replace clinical judgment, once cases are winnowed to manageable diagnostic groups, point of care interventions plus electronic reminders can be triggered which allow further stratification of groups. In short, these algorithms formed the “open book” approach: get the disease management program to page one and help write the ending. By focusing care within one hospital day on high likelihood cases, the present invention makes possible processes to influence provider performance, enhance documentation, optimize quality of care received, and assist in determination of a final discharge diagnosis.

In order to rapidly identify patients with congestive heart failure, pneumonia and acute myocardial infarction, the computer algorithms were run on a once to twice daily basis. The 12-24 hour cycle allowed sufficient time for identification, clinician follow-up, manually-triggered electronic alerts, and physician detailing. The present invention works with common databases, such as Access 98, to import, query, generate reports, and track the inpatients because the sophisticated algorithms are embedded in a portable, scalable program. This allows any medical center with access to flat files of laboratories, admission complaints, and medications to do highly effective patient tracking of leading diagnoses. The program can also be scaled up to Access 2003 and shared over the web. Thus, it is possible for the program to be shared by over a dozen real time users with reports going out to every inpatient case manager, nurse manager, and health information technician, in addition to the clinical performance improvement team.

The present invention is also able to prepare alerts at point of care to the clinicians on their next set of rounds. Honing the data dictionary for the common triage complaints increased sensitivity, as many patients presented with conditions without specific connection to the disease condition, such as “weakness.” Adding “parsing” to the diagnosis/complaint groups further improved the sensitivity, allowing partial matches, abbreviations and misspelled items. Finally, establishing a medication-based approach allowed a means by which clinical practice could be gauged and secondary diagnoses captured, though physicians may have missed the diagnosis in the list of key active conditions. Because secondary diagnoses are a cause of both valid coding decisions as well as coding errors, this enabled a concurrent process for improving documentation to keep it in line with clinical practice and in turn populate the claims database with accurate information.

1. A computer-implemented method of tracking patients with target clinical conditions, the method comprising the steps of:
a. scoring diagnostic or complaint text fields;
a. scoring medications administered to patients;
b. scoring patient laboratory test data; and
c. sorting the scored patient data into groups of suspected clinical conditions.

2. The method of claim 1, wherein said target clinical conditions are selected from the group consisting of cardiac conditions and pneumonia.

3. A computer-implemented method of tracking heart failure, acute myocardial infarction, and community-acquired pneumonia patients, the method comprising the steps of:
a. computer-screening patient medications prescribed;
b. counting classes of medications per patient;
c. tabulating groups of medications into scores;
d. computer-screening patient chief complaints or diagnoses;
e. checking said chief complaints or diagnoses against a set of standard terms;
f. checking said chief complaints or diagnoses against a set of partial terms;
g. flagging patients with a score indicating possible diagnosis;
h. computer-screening patient laboratory values;
i. wherein, if said laboratory values are above a certain threshold, patients are sorted into certain diagnoses, if said laboratory values are very low, said diagnoses are excluded, if said laboratory values are moderately high, the system requires additional information such as medications or diagnosis scores; and
j. combining all three scores to create a list of patients with suspected acute myocardial infarction, pneumonia, and heart failure.

4. A computer-implemented method of identifying heart failure patients, the method comprising the steps of:
a. computer-screening patient admission records for one or more specific heart failure terms;
b. computer-screening patient laboratory records for BNP levels above about 400 ng/dl;
c. computer-screening patient admission records for one or more terms suggestive of heart failure and patient laboratory data for BNP levels above about 100 ng/dl;
d. computer-screening patient medication records for one or more heart failure specific medications and laboratory records for BNP levels above about 100 ng/dl;
e. computer-screening patient medication records for two or more classes of medications suggestive of heart failure and laboratory records for BNP levels above about 100 ng/dl; and
f. computer-screening patient medication records for three or more classes of medications suggestive of heart failure.

5. The method of claim 4, wherein said one or more specific heart failure terms are selected from the group consisting of: heart, failure, pulmonary edema, and CHF.

6. The method of claim 4, wherein said one or more terms suggestive of heart failure are selected from the group consisting of: dyspnea, shortness of breath, SOB, shortness, congestion, congest, cardiomyopathy, cardio, edema, weakness, volume overload, renal insufficiency, and atrial fibrillation.

7. The method of claim 4, wherein said heart failure specific medications are selected from the group consisting of: Carvedilol, Digoxin, Aldactone, and Eplerenone.

8. The method of claim 4, wherein said classes of medications suggestive of heart failure are selected from the group consisting of: angiotensin converting enzymes, beta blockers, cardiac glycosides, angiotensin receptor blockers; and heart failure diuretics.

9. The method of claim 4, further comprising the step of:
a. computer-screening patient laboratory records for troponin I levels above about 0.4 mg/dl within 24 hours, thereby identifying patients with acute myocardial infarction.

10. A computer-implemented method of identifying community-acquired pneumonia patients, the method comprising the steps of:
a. computer-screening patient admission records for one or more specific community-acquired pneumonia terms; and
b. computer-screening patient admission records for one or more terms suggestive of community-acquired pneumonia and medication records containing one or more community-acquired pneumonia specific medications;
c. wherein, if said term suggestive of community-acquired pneumonia is “fever,” a medication record containing two or more community-acquired pneumonia specific medications are required.

11. The method of claim 10, wherein said community-acquired specific pneumonia terms are selected from the group consisting of: cough and fever, and pneumonia.

12. The method of claim 10, wherein said terms suggestive of community-acquired pneumonia are selected from the group consisting of: cough, bronch, CAP, pleuritic pain, lower resp, and fever.

13. The method of claim 10, wherein said community-acquired pneumonia specific medications are selected from the group consisting of: β-lactams: cefepime, imipenem, meropenem, piperacillin, tazobactam, cefuroxime, cefpodoxime, cefprozil, amoxicillin, clavulanate; macrolides: erythromycin, azithromicyn, clarithromycin; cefotaxime, ceftriaxone, ertapenem, sulbactam, ampicillin; doxocycline, antipneumococcal fluoroquinolones: ciprofloxacin, ofloxacin, levofloxacin, sparfloxacin, gatifloxacin, moxifloxacin, trovafloxacin, gemifloxacin; aminoglycosides, nonpseudomonal fluoroquinolones; vancomycin, clindamycin; methicillin, linezolid; ketolides: telithromycin; doxocycline, aztreonam.

14. A computer-implemented method of identifying heart failure, pneumonia, and acute myocardial infarction patients, the method comprising the steps of:
a. computer-screening patient admission records for one or more specific heart failure terms;
b. computer-screening patient laboratory records for BNP levels above about 400 ng/dl;
c. computer-screening patient admission records for one or more terms suggestive of heart failure and laboratory records containing BNP levels above about 100 ng/dl;
d. computer-screening patient medication records for one or more heart failure specific medications and laboratory records containing BNP levels above about 100 ng/dl;
e. computer-screening patient medication records for two or more classes of medications suggestive of heart failure and patient laboratory records for BNP levels above about 100 ng/dl;
f. computer-screening patient medication records for three or more classes of medications suggestive of heart failure;
g. computer-screening patient laboratory records for troponin I levels above about 0.4 mg/dl within 24 hours;
h. computer-screening patient admission records for one or more specific community-acquired pneumonia terms; and
i. computer-screening patient admission records for one or more terms suggestive of community-acquired pneumonia and medication records for one or more community-acquired pneumonia specific medications; wherein, if said term suggestive of community-acquired pneumonia is “fever,” a medication record containing two or more community-acquired pneumonia specific medications is required.
